(19) United States
(12) Patent Application Publication
(10) Pub. No.: US 2017/0000369 A1
(43) Pub. Date: Jan. 5, 2017

(54) ELECTROCARDIOGRAM SYSTEMS AND RELATED METHODS

(71) Applicant: ELWHA LLC, BELLEVUE, WA (US)

(72) Inventors: Roderick A. Hyde, Redmond, WA (US); Elizabeth A. Sweeney, Seattle, WA (US)

(21) Appl. No.: 14/791,318

(22) Filed: Jul. 3, 2015

(51) Int. Cl. A61B 5/0408; A61B 5/00

(52) U.S. Cl. CPC A61B 5/04085; A61B 5/6806; A61B 5/7405; A61B 5/442; A61B 5/0077; A61B 5/7455; A61B 2562/0219

(57) ABSTRACT

Embodiments disclosed herein are directed to a system for measuring an electrical potential at one or more positions on a subject and determining if the electrical potential measurements are taken at one or more selected positions, and methods of using the same. The system can include at least one glove having at least one electrode thereon configured to collect electrical potential measurements and at least one position sensor configured to collect position data to determine if the electrical potential measurements are taken at a specified location on the subject.

### RELATED METHODS

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### BACKGROUND

Electrocardiogram (EKG) systems are typically utilized to detect and record electrical activity of a heart of a subject over a period of time. For example, an EKG system detects a voltage between electrical potentials detected at two or more surfaces of a subject. The voltage can measure a certain angle of the electrical axis of the heart or an anatomical area of the heart.

Typically, EKG systems are used with a 12 lead EKG. The 12 lead EKG measures voltage at twelve different angles of the electrical axis of the heart. In a conventional 12 lead EKG, ten separate and distinct electrodes are placed on ten different and specific surfaces of the subject simultaneously. Each of the ten separate and distinct electrodes are connected to a computing device. Each of the ten separate and distinct electrodes detect electrical potentials at a corresponding surface of the subject.

The computing device uses the detected electrical potentials to determine twelve different voltages. For example, the computing device can determine the three limb leads (e.g., limb I, limb II, and limb III), the three augmented limb leads (e.g., aVR, aVL, and aVF), and the six precordial leads (e.g., the voltage between the six chest electrode placements and Wilson's central terminal). The term “lead” in electrocardiography in the context of what the computing device determines refers to the 12 different vectors along which the heart's depolarization is measured and recorded.

The computing device then generates a report including a graphical representation of the voltage of each lead as a function of time. Typically, each lead has a specific location on the generated report.

### SUMMARY

Embodiments disclosed herein are directed to a system for measuring the electrical potential at one or more positions on a subject and determining if the electrical potential measurements are taken at one or more desired positions, and methods of using the same. In an embodiment, a system for capturing electrocardiogram readings is disclosed. The system includes at least one glove including at least one electrode configured to measure one or more electrical potential measurements of a subject and at least one position sensor configured to measure one or more positional measurements of the at least one electrode relative to the subject. The system further includes a controller operably coupled to the at least one electrode and the at least one position sensor. The controller includes a processor configured to determine if the at least one electrode is at or near a preferred measurement site for measuring the one or more electrical potential measurements at least partially based on the one or more positional measurements.

In an embodiment, a method of determining proper placement of at least electrode of at least one glove for determining electrical characteristics of a cardiovascular system of a subject is disclosed. At least one glove is provided, which includes the at least one electrode configured to measure one or more electrical potential measurements of a subject and at least one position sensor configured to determine a position of the at least one electrode relative to the subject. One or more positional measurements are collected using the at least one position sensor. Each of the one or more positional measurements is correlated with at least one reference location stored in memory. The at least one reference location includes at least one preferred measurement site. An indication is provided regarding a position of the at least one electrode relative to the at least one preferred measurement site.

Features from any of the disclosed embodiments can be used in combination with one another, without limitation. In addition, other features and advantages of the present disclosure will become apparent to those of ordinary skill in the art through consideration of the following detailed description and the accompanying drawings.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic plan view of a glove configured to capture one or more electrocardiogram measurements and determine a relative position of one or more portions of the glove, according to an embodiment.

FIG. 2 is a schematic of an electrocardiogram system in use, according to an embodiment.

FIG. 3 is a schematic of an electrocardiogram system in use, according to an embodiment.

FIG. 4 is a schematic of an electrocardiogram system in use, according to an embodiment.

FIG. 5 is a schematic diagram of an electrocardiogram system according to an embodiment.

FIG. 6 is a flow chart of a method of determining proper placement of sensors of at least one glove for determining electrical characteristics of a cardiovascular system of a subject, according to an embodiment.

### DETAILED DESCRIPTION

Embodiments herein are directed to a system configured to collect electrocardiographic information from preferred measurement positions on a subject and methods of using the same. The system includes at least one glove having one or more position sensors, one or more electrodes, and a controller operably coupled to and for controlling the one or more position sensors and the one or more electrodes. The system utilizing at least one gloves having the one or more electrodes and the one or more position sensors can be used to provide accurate measurements of electrical potential at any point on a subject's body and determine if such measurement was taken at a preferred measurement site. The system herein can be used to measure electrical potential at any position on a subject and determine whether said position is a preferred position for collecting accurate EKG data.

Electrocardiograms (commonly referred to as an ECG or EKG) can indicate relative differences in heart rhythm based on measured electrical activity of the heart from different angles or leads. EKGs include electrical potentials measured along different angles or leads related to the electrical axis of the heart. Leads include limb leads, augmented limb leads, and precordial leads. Each lead can provide different information about the electrical activity of the heart of a subject. For example, the lead between the left arm and right arm detected by a positive electrode at the left wrist and negative electrode at the right wrist can collect information about the voltage along the angle therebetween. Similarly, the lead between the right arm and left leg detected by a positive electrode at the left ankle and negative electrode at the left wrist can collect information about the voltage along the angle therebetween. The positioning of each electrode can vary but should remain in positions to measure voltage across the 12 leads (e.g., angles) with respect to the heart. For instance, the right wrist and left wrist electrodes can be positioned on the right shoulder and left shoulder respectively and still maintain substantially in the same angular relation to the heart. Accurate positioning of each electrode in each lead is vital to collecting accurate and dependable EKG data. The sum of the electrical potentials from each of the 12 leads is used to construct an EKG. However, such a positioning is normally carried out with 10 separate electrodes placed in a static position on a subject and wired to a monitor.

A system utilizing one or more gloves having one or more electrodes and position sensors thereon can be used to provide accurate measurements of electrical potential at any point on a subject's body and determine if such measurement was taken at a preferred measurement site. In an embodiment, preferred measurement sites have utility as an electrode placement site for a lead and provide accurate and useful measurements of electrical potential of the subject's heart. In an embodiment, the preferred measurement sites are the most efficient locations to obtain the desired information. For example, suitable preferred measurement sites can include any of the traditionally used lead locations such as limb lead locations, augmented limb lead locations, precordial lead locations; or can include locations therebetween such as points between traditional lead locations, but which can supply substantially the same electrical information for the corresponding lead. Such locations between traditional lead locations can provide a preferred measurement site in which the electrical potential measurement corresponding to the particular lead (e.g., angle) is strong enough to provide reliable data (e.g., a right shoulder wherein a right wrist may be the traditional lead location). In an embodiment, the preferred measurement site can further include a region surrounding a traditional lead location, such as a region within about 15 cm of the traditional lead location, within about 10 cm, within about 5 cm, or within about 2 cm of the traditional lead location. In an embodiment, a preferred measurement site can include a site having an electrical potential measurement above a selected threshold (e.g., a magnitude of the measured electrical potential is above a selected amount) or an electrical potential measurement matching a selected pattern (e.g., a predicted electrical pattern such as a time-sequenced electrical potential corresponding to the QRS complex of an EKG, the PR interval of an EKG, the QT interval of an EKG, or other suitable electrical potential measurement). The one or more gloves can be moved from one lead position to another lead position to gather electrical potential measurements without the need of having static electrodes placed on the subject. The position sensors on the one or more gloves can collect position data to ensure that the glove or electrodes thereon are at a preferred measurement site (e.g., lead site).

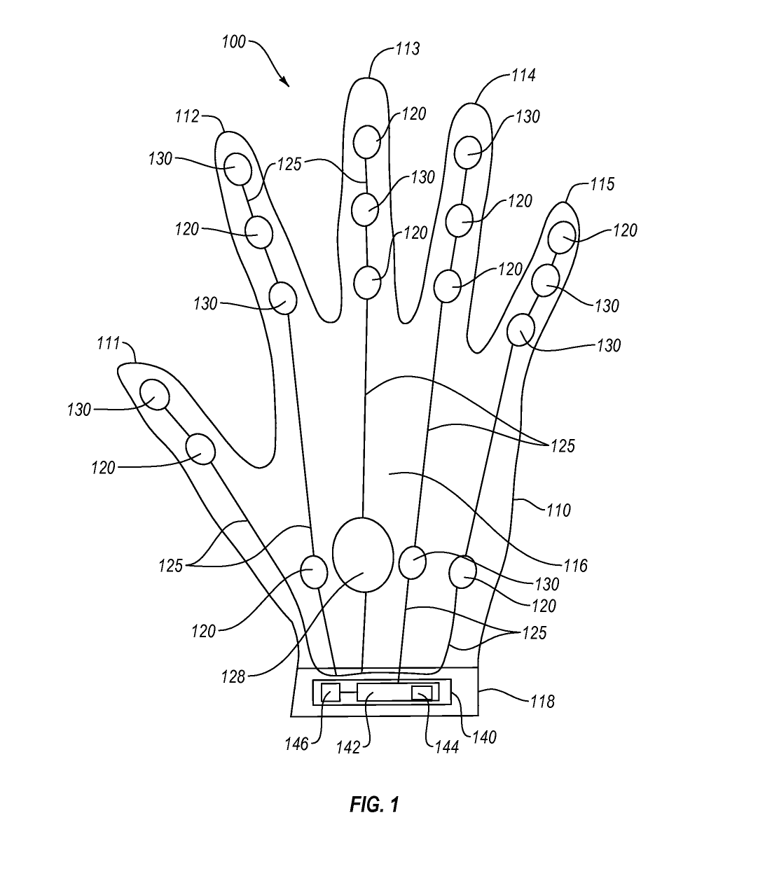

FIG. 1 is a schematic plan view of an electrocardiogram system 100 for collecting EKG measurements, according to an embodiment. The system 100 includes at least one glove 110 and a controller 140. The at least one glove 110 includes one or more sensors thereon, such as one or more sensors facing outward from the palm side of the glove 110. The one or more sensors can include at least one position sensor 120 and at least one electrode 130. The at least one position sensor 120 can be configured to measure, capture, or collect one or more positional measurements of one or more of the at least one position sensor 120 or the at least one electrode 130 relative to the body of the subject. The at least one electrode 130 can be configured to measure one or more electrical potential measurements. Each of the one or more position sensors 120 and the at least one electrode 130 can be operably coupled to the controller 140 and configured to transmit measured data (e.g., positional measurements or electrical potential measurements) to the controller 140 via electrical connections 125 or a wireless connection. The controller 140 can include a processor 142 and memory 144 configured to correlate, compare, process, store, output the measured data, or output information (e.g., an EKG or indication that the at least one electrode is at a preferred measurement position) based on the measured positions or electrical potentials. The controller 140 can be configured to correlate the electrical potential measurements with the positional measurements corresponding thereto. The controller 140 can be configured to process the measured electrical potentials and positional information corresponding thereto to derive an EKG therefrom.

The glove 110 can include a central portion or palm 116, a wrist portion 118, and a sheath for one or more fingers of a hand of a user. For the purposes of this application, and unless otherwise indicated herein, the thumb is referred to as a finger. In an embodiment, the glove 110 can include five fingers or finger sheaths 111-115; one for each finger of the hand. In an embodiment, the glove 110 can include less than five sheaths, such as 1-4 sheaths, two sheaths, or 3 sheaths. In an embodiment, the sheaths can be only configured as the thumb, index finger, or little finger. In an embodiment, the sheaths can be configured only for the thumb and index finger. One or more sensors 120 or electrode 130 can be disposed one each sheath, the palm, or the back of the glove 110.

The glove 110 can be made of one or more materials, such as natural materials (e.g., leather, cotton, rubber, silk, etc.) or man-made materials (e.g., polyester, nylon, rayon, latex, etc.). For example, the glove 110 can be made of cotton material housing one or more sensors embedded therein or thereon. In an embodiment, the glove can include a plurality of materials, such as cotton material on one or more portions of the glove and a manmade material one or more portions of the glove. For example, the glove 110 can include cotton material on the back and a nylon material on the palm. In an embodiment, one or more rubber or latex portions configured to house the one or more sensors can be disposed at one or more locations on the glove. The one or more rubber or latex portions can be located around each sensor to substantially isolate each sensor to reduce or eliminate interference (e.g., electrical interference) from adjacent sensors.

In an embodiment, each finger sheath 111-115 of the glove 110 can include one or more sensors (e.g., the one or more position sensors 120 and at least one electrode 130) therein or thereon. One or more of the finger sheaths 111-115 can include one or more sensors, such as one sensor, two sensors, or three sensors or more. For example, each sheath can include at least one position sensor 120 and at least one electrode 130. The thumb sheath 111 of the glove 110 depicted in FIG. 1 can include an electrode 130 in a distal portion and a position sensor 120 at a proximal portion. In an embodiment, the electrode 130 can be disposed in the proximal portion and the position sensor 120 in the distal portion or vice versa. Each of the electrode 130 and the position sensor 120 in the thumb sheath 111 can be operably coupled to the controller 140 via an electrical connection 125 or a wireless connection. In an embodiment, the finger sheaths 112-115 can each include one or more sensors therein or thereon. For example, the index finger sheath 112 can include an electrode 130 at a proximal end (e.g., in the finger tip), a position sensor 120 in the medial portion, and another sensor such as another electrode 130 in the proximal portion. In an embodiment, the one or more sensors in a sheath, such as the middle finger sheath 113, can include a position sensor 120 at a proximal end, an electrode 130 in the medial portion, and another sensor such as another position sensor 120 in the proximal portion. The one or more sensors in each of the ring finger sheath 114 or the little finger sheath 115 can be similar or identical to any of the sensor configurations disclosed herein. For example, the sensors in each of the finger sheaths 111-115 can be similar or identical to each other, such as each finger sheath 112-115 having an electrode 130 at the distal portion, a position sensor 120 at the medial portion, and another position sensor 120 at the proximal portion thereof. In an embodiment, each finger sheath 112-115, and optionally thumb sheath 111, can include an electrode 130 at the medial portion, a position sensor 120 at the distal portion, and another position sensor 120 at the proximal portion. In an embodiment, any other configuration of the sensors and electrodes can be used, such as all electrodes 130 on one or more finger sheaths, or all position sensors on one or more finger sheaths, or a combination thereof, such as alternating finger sheaths having all electrodes 130 and all position sensors 120. In an embodiment, the palm 116 can include one or more of the at least one electrode 130 or the at least one position sensor 120.

In an embodiment, the thumb sheath 111 can include a different number or type of sensors from the finger sheaths 112-115. For example, the thumb sheath 111 can include two sensors and the fingers sheaths 112-115 can include three sensors. In an embodiment, one or more of the sensors in the finger sheaths 112-115 can include identical sensors (e.g., position sensors or electrodes) in identical locations (e.g., distal, medial, or proximal). In an embodiment, one or more of the sensors in the finger sheaths 112-115 can include different sensors (e.g., position sensors or electrodes) in identical locations (e.g., distal, medial, or proximal). In an embodiment, the thumb sheath 111 can include different sensors than the finger sheaths 112-115.

In an embodiment, one or more position sensors 120 or electrodes 130 can be disposed on the central portion (e.g., palm 116 or back) of the glove 110. In an embodiment, the palm 116 of the glove can accommodate a larger sensor 128. For example, the glove 110 can include the larger sensor 128. The larger sensor 128 can include at least one of a position sensor 120 or electrode 130 having a larger footprint than the sensors in the finger sheaths 111-115, which can provide increased sensitivity over similar but smaller sensors disposed in the finger sheaths. In an embodiment, the sensors 120, 128, or 130 in the palm 116 can be operably coupled to the controller 140 through one or more the electrical connections 125 or a wireless connection. In an embodiment, the finger sheaths 111-115 can include one or more position sensors 120 therein or thereon and the palm 116 can include one or more electrodes 130. The plurality of position sensors 120 in the finger sheaths 111-115 can substantially ensure that the position of the at least one electrode 130 on the palm 116 is satisfactorily known at least by the relationship between the plurality of position sensors 120 on the finger sheaths 111-115 and the palm 116. In an embodiment, the finger sheaths 111-115 can include one or more electrodes 130 therein or thereon and the palm 116 can include one or more position sensors 120 therein or thereon. The position of the palm 116 can indicate the relative position of the electrodes 130 on the finger sheaths 111-115 as related to a preferred measurements site measured by the position sensors 120 on the palm 116.

In an embodiment, each position sensor 120 can include a corresponding electrode 130 such that each position sensor 120 indicates the position of a specific electrode 130. For example, each finger sheath 111-115 can include one position sensor 120 (e.g., at a distal end or medial portion) and one corresponding electrode 130 (e.g., at a medial portion or distal end), where a spatial relationship between the position sensor 120 and the corresponding electrode 130 is known such that the position of the electrode 130 can be determined by the measured positional information from the position sensor 120 as related to the spatial relationship. In an embodiment, an electrode 130 located on a surface-contacting portion of the glove (e.g., the palm, or the inner surface of a finger) can be accompanied by a corresponding position sensor 120 on an opposite, non-surface contacting portion of the glove (e.g., the back of the glove opposite the palm, or the back of a finger opposite the location of finger electrode 130).

In an embodiment, one or more position sensors 120 or electrodes 130 can be positioned on any surface of the glove, such as adjacent to the palm of the glove 110 or the back of the glove 110. The one or more sensors can be embedded within the glove 110, such as between one or more layers of the glove 110. In an embodiment, one or more sensors 120 or electrodes 130 can be at least partially embedded within the glove 110 and have one or more portions protruding therefrom, such as an electrode partially embedded within the glove 110 and having a portion thereof protruding from the glove 110 at a portion of the glove 110 designed to contact the subject (e.g., the palm side of the finger sheaths 111-115 or the palm 116).

In an embodiment, more than one type of position sensor 120 can be included in one or more portions of the glove 110. For example, a first type of position sensor (e.g., accelerometer) can be positioned in the palm 116 or one or more of the finger sheaths 111-115 and a second type of position sensor (e.g., contact sensor, topography sensor, optical sensor, etc.) can be positioned in one of the finger sheaths 111-115 or the palm 116. Multiple types of position sensors 120 can be used to provide multiple positioning functionalities such as by relative movement, imaging, topography, or other position identifying means. Such embodiments can provide greater positional accuracy than embodiments having only one type of position sensor.

The at least one position sensor 120 can include one or more of an accelerometer, a topography sensor, an optical sensor, an acoustic sensor, a contact sensor, a micro-impulse radar, or any other sensor suitable for collecting data related to the position of the glove 110 or a discrete portion thereof. For example, the at least one position sensor 120 can include at least one accelerometer configured to detect relative movement or orientation (e.g., direction of tilt or direction that the glove 110 is facing) of the glove 110 or a discrete portion thereof over time, such as from a first time to a second time. The accelerometer can be configured to determine the relative orientation of the glove 110 or a discrete portion thereof. In an embodiment, more than one accelerometer can be used on the glove 110 including at least one accelerometer for determining changes in relative position and at least one accelerometer for determining the relative orientation of the glove 110 or a discrete portion thereof. In such an embodiment, the position sensors 120, including accelerometers, can be used determine the position and orientation of each portion of the glove 110 including the finger sheaths 111-115 thereon. When the starting position of the glove 110 or a portion thereof is known, the subsequent position of the glove 110, with respect to a subject, can be determined using only the movement and orientation information collected from the at least one accelerometer. For example, the controller 140 can relate any measured movement and orientations from the starting position in a chronological order to determine a route and final destination of the glove 110 over a period of time. In an embodiment, the at least one accelerometer can be configured to determine the acceleration or velocity of the glove 110 or a portion thereof (e.g., an electrode) at the time of measuring the electrical potential at a location on a subject, and transmit the same to the controller 140. The controller 140 can be used to determine if the at least a portion of the glove 110 was moving at a velocity, or accelerating below a threshold velocity or acceleration at the time that one or more measurements (e.g., electrical potential measurements or positional measurements) were made. In instances where the glove 110 or a portion thereof moving faster than the threshold velocity or acceleration, the controller 140 can tag or otherwise indicate that such measurements (e.g., electrical potential or acoustic measurements) were made at too high of a velocity or acceleration to ensure reliable results. In instances where the glove 110 was making insufficient contact with the subject when the measurement was taken, the controller 140 can tag or otherwise indicate that such measurements were made with insufficient contact and delete or otherwise segregate such data from calculations or determinations. For example, in an embodiment, the controller 140 can be configured to delete from the memory 144 all measurements taken when the corresponding velocity is about 2 millimeters per second (mm/s) or more, such as about 4 mm/s, about 8 mm/s, about 10 mm/s to about 50 mm/s, or about 10 mm/s. Similar operations can be carried out with acceleration data. For example, only measurements taken when the acceleration of the accelerometer is below about 9.8 m/s², such as about 8 m/s², about 5 m/s², or about 2 m/s² may be stored in the memory or tagged.

In an embodiment, the at least one position sensor 120 can include a topography sensor configured to sense the topography of the skin of the subject. For example, the topography sensor can include a video camera or infrared camera configured to capture one or more images of the skin of the subject. The captured video or infrared images of the topography of the skin of the subject can be transmitted to the controller (e.g., the memory 144) wherein the images can be compared to a topographical atlas (e.g., an atlas including one or more reference images of the physiological features or biological structures capable as serving as references points of the subject or general demographic) or database of reference images to determine a relative position via a match between the topographical features in the captured images and the topographical atlas or database of reference images.

In an embodiment, the at least one position sensor 120 can include an optical sensor. The optical sensor can include an imaging sensor such as a video camera or a near infrared sensor configured to collect infrared image data of the position of one or more biological features of a subject (e.g., bones, subsurface blood vessels, muscles, etc.). The infrared sensor can be specifically configured to capture images of blood vessels or other biological features of the subject. The infrared sensor can transmit the infrared image data to the controller 140. The controller 140 can be used to determine a correlation between the collected infrared images and previously collected images or an anatomical atlas (e.g., atlas including one or more reference images of physiological features or biological structures capable as serving as references points, specific to the subject or a general demographic) to determine a match or proximity to a match of anatomical structures in the images and atlas to the relative position of the glove 110 at a point in time. For example, the reference images of the atlas can include one or more reference images of the blood vessels (e.g., blood vessel reference images) of the subject or a general demographic and the captured images of the blood vessel can be compared to the blood vessel references images to determine a match in position therebetween, such as by the controller 140 or processor 142.

In an embodiment, the at least one position sensor 120 can include an acoustic sensor configured to collect acoustic data (e.g., acoustic images) of the position of one or more biological structures of the subject. The acoustic sensor can include an ultrasonic (ultrasound) sensor or receiver configured to collect ultrasonic data (e.g., ultrasound/ultrasonic images) of the position of one or more biological structures of the subject. The ultrasonic sensor can transmit ultrasonic data to the controller 140. The controller 140 can be used to determine a correlation between the collected ultrasonic data and previously collected data or the anatomical atlas (e.g., reference ultrasonic images) to determine a match or proximity to a match of anatomical structures in the images to determine the relative position of the glove 110 at a point in time.

In an embodiment, the at least one position sensor 120 can include an audio sensor configured to detect audio indications of heart beat or blood flow of the subject. For example, the audio sensor can include a sound detector or microphone configured to detect heart beat or can include an ultrasound Doppler sensor to detect blood flow in the subject. The sound detector can be configured to detect and record heart beat data or blood flow data which can be transmitted to the controller 140. The heart beat data or blood flow data can be used to correlate the recording position by comparison to previously recorded or known data corresponding to a particular position.

In an embodiment, the at least one position sensor 120 can include a micro-impulse radar. The micro-impulse radar can be configured to collect data of the position of one or more of external surface structures (e.g., one or more contours of the skin) or internal (subsurface) structures (e.g., bone, muscle, vessels, etc.) of a subject relative to the one or more electrodes 130 or the micro-impulse radar. The micro-impulse radar can transmit the micro-impulse radar data to the controller. The controller 140 can be used to determine a correlation between the collected micro-impulse radar data and previously collected data (e.g., one or more images) or the anatomical atlas to determine a match or proximity to a match of anatomical structures in the images to determine the relative position of the glove 110 at a point in time.

In an embodiment, the at least one position sensor 120 can include one or more contact sensors. The one or more contact sensors can include any one of a pressure sensor (e.g., piezoelectric sensor), and electro-magnetic sensor, a piezoresistive sensor, a capacitive sensor, an elastoresistive sensor, a stress sensor, an ultrasonic transducer, or an electrical resistance sensor, each configured to collect data on the amount of pressure on the pressure sensor, which can indicate the level of contact of one or more portions of the glove 110 with a subject. The pressure data can be collected and transmitted to the controller 140. The controller 140 can be configured to determine if the at least a portion of the glove 110 was contacting the subject with a selected amount of pressure at the time that one or more measurements (e.g., electrical potential measurements or positional measurements) were made. In instances where the glove 110 was making sufficient contact with the subject, the controller 140 can be configured to tag or otherwise indicate that such measurements (e.g., electrical potential or acoustic measurements) were made with sufficient contact to ensure reliable results. In instances where the glove 110 was making insufficient contact with the subject when the measurement was taken, the controller can be configured to tag or otherwise indicate that such measurements were made with insufficient contact and delete or otherwise segregate such data from calculations or determinations. For example, in an embodiment, the controller 140 can be configured to delete from the memory 144 all electrical potential measurements taken when the corresponding pressure on an adjacent pressure sensor was below about 7 kPa, below about 35 kPa, below about 70 kPa, or outside the range of 7 kPa to about 140 kPa. Such pressure thresholds can ensure that the corresponding electrical potential measurements were taken when the electrode 130 corresponding to the pressure sensor had sufficient contact to allow an accurate measurement of electrical potential.

The at least one electrode 130 can include any electrode configured to sense electrical potential through the dermis of a biological subject, such as solid gel electrodes, cloth electrodes, tape electrodes, foam electrodes, cup electrodes, etc. In an embodiment, one or more of the at least one electrode 130 can be a negative electrode or a positive electrode and can be selectively activated or have data selectively transferred therefrom responsive to the processor determining that the position of the at least one electrode is suitable for a negative or a positive electrode. In an embodiment, one or more of the at least one electrode 130 can be switchable between a negative electrode or a positive electrode and can be selectively switched responsive to the processor determining that the position of the at least one electrode is suitable for a negative or a positive electrode. In an embodiment, a conducting gel can be provided in or on the at least one electrode 130. In an embodiment, each electrode 130 can be of identical size in the system 100. In an embodiment, some of the electrodes 130 can be a different size as any of the other electrodes 130 in the system 100. For example, the glove 110 can include a large sensor 128 configured as an electrode 130 and having a larger surface area than adjacent electrodes 130 such as those in the finger sheaths 111-115. In an embodiment, the large sensor 128 can exhibit a surface area of at least two times that of the electrodes 130 or position sensors 120 adjacent thereto, such as about two times to about four times larger, or about three times larger.

Each of the at least one position sensor 120 and the at least one electrode 130 can be configured to transmit the measured data to the controller 140 (e.g., to the memory 144 or processor 142) upon collecting said data. The controller 140, or the at least one position sensor 120 and at least one electrode 130 can be configured to time stamp or tag each electrical potential measurement or positional measurement with the time that each measurement was taken. In an embodiment, the controller 140 can be configured to tag (e.g., time stamp) and correlate each electrical potential measurement with each corresponding positional measurement (e.g., position, pressure, velocity, or acceleration). In such embodiments, the changes in electrical potential and position of the glove 110 can be accurately measured over time (e.g., in a time sequence).

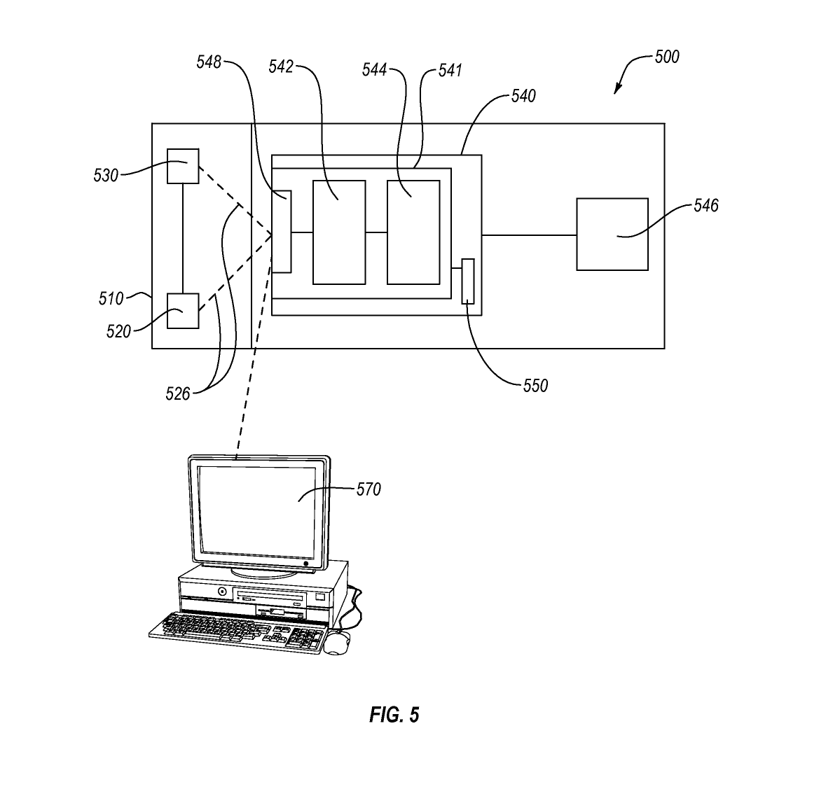

The controller 140 can be positioned on any portion of the glove 110 which does not interfere with the functioning of the position sensors 120 or the electrodes 130. In an embodiment, it may be desired to position the controller 140 on a portion of the glove 110 distant from the measurement surfaces, such as to reduce or eliminate any interference with the sensors. For example, the controller 140 can be positioned in the wrist portion 118 of the glove 110. The wrist portion 118 can be positioned proximate to or at least partially extend over the wrist of the user. In an embodiment, the controller 140 can be positioned on the back of the central portion of the glove (e.g., substantially opposite the palm 116). In an embodiment, the controller 140 can be remote from the glove 110 (e.g., in a desktop computer) and communicate with the one or more sensors wirelessly. The controller 140 includes the processor 142 and the memory 144. As discussed in more detail below, in an embodiment the controller 140 can include at least one indicator 146. As explained in more detail below, the controller 140 can include a communications relay (FIG. 5) configured to communication with one or more of the electrodes 130, the position sensors 120, or a remote device (e.g., an associated computing device, a display, additional glove 110, etc.).

The processor 142 can be configured to determine if the at least one position sensor 120 or electrode 130 is at or near a preferred measurement site. The processor 142 can be configured to associate (e.g., tag a correspondence between) each of the one or more electrical potential measurements with a corresponding one of the one or more positional measurements. The processor 142 can be configured to compare at least one or the one or more electrical potential measurements or the one or more positional measurements with corresponding measurements associated with the preferred measurement sites and determine if the at least one electrode 130 is at a preferred measurement site base thereon. For example, the processor 142 can be configured to compare one or more measured or captured images (e.g., internal or external images) with one or more reference images to determine the relative position of the glove 110, position sensor 120, or electrode 130, relative thereto. The processor 142 can be configured to cause the indicator 146 to provide one or more indications (e.g., visual, audio, or tactile) that the glove or a portion thereof is at a specific preferred measurement site or not at the specific preferred measurement site, responsive to the comparison above. In an embodiment, the processor 142 can be configured to reject or delete one or more electrical potential measurements that do not correspond to one of the preferred measurement sites. The processor 142 can be operably coupled to the memory 144 to access measurements, data, or program instructions stored thereon.

The electrical potential measurements, positional measurements, preferred measurement sites, time data, database of reference images or anatomical atlases, program instructions for correlating or analyzing any of the same, or program instructions for indicating if the glove 110 is at a preferred measurement site or not at a preferred measurement site can be stored in the memory 144. The memory 144 can include a non-transitory computer readable medium such as a hard drive, disk, solid state memory device, or other memory storage medium. The memory 144 can be operably coupled to one or more of the processor 142, the at least one electrode 130, the at least one position sensor 120 (e.g., either directly or indirectly through the processor 142), or the indicator 146.

The controller 140 can include at least one indicator 146. The at least one indicator 146 can include one or more of an audio indicator, visual indicator, or tactile indicator. The processor 142 can direct the at least one indicator to provide one or more indications to the subject or user. In an embodiment, an audio indicator can include a chime, speaker, or other audio output device configured to provide an one or more differing audible indications to a subject or user of the glove 110. The audio indicator can be configured to provide a first indication (e.g., first tone) if the glove 110 or specific position sensor 120 or electrode 130 thereon is at or near a preferred measurement site and a second indication (e.g., a second, different tone) when the glove 110 or specific position sensor 120 or electrode 130 is not at or near the preferred measurement site. In an embodiment, the audio indicator can be configured to provide an audio indication at a slower pace (e.g., larger spacing between each tone of a series of tones) when the glove 110 or specific position sensor 120 or electrode 130 thereon is at or near a preferred measurement site and to speed up the audio indication to a faster pace when the glove 110 or specific position sensor 120 or electrode 130 thereon is at or near a preferred measurement site. The processor 142 can be configured to direct the audio indicator to provide the differing indications described above, such as to steer the user to one or more preferred measurement sites.

In an embodiment, the visual indicator can include one or more lights (e.g., an array) or a screen (e.g., an LED screen) associated with the glove 110 (e.g., on the glove 110 or separate therefrom). The one or more lights can be positioned on one or more portions of the glove 110 visible to a user during use, such as on the wrist portion 118, the back of the glove 110 (e.g., the opposite side of the glove 110 from the palm 116), the backs of the finger sheaths 111-115, or on a device remote from the glove 110 such as an associated screen or computing device. The one or more lights can include one or more lights having different colors or intensities. For example, a red light can indicate that the glove 110 or specific position sensor 120 or electrode 130 thereon (e.g., on a specific finger sheath) is not near a preferred measurement site, while a different colored light, such as green, can indicate that the glove 110 or specific position sensor 120 or electrode 130 thereon is near the preferred measurement site. In an embodiment, the visual indicator can be configured to strobe at a faster or slower pace depending on the proximity of one or more portions of the glove 110 to a preferred measurement site. For example, the visual indicator can strobe at a slower pace while the glove 110 is farther from the preferred measurement site and speed up to a faster pace as the glove 110 is moved closer to the preferred measurement site.

In an embodiment, the indicator can include an array of lights. The array of lights can include three or more adjacent lights. The array can be configured such that more of the lights in the array light up when the glove 110 or specific position sensor 120 or electrode 130 is at or near a preferred measurement site. In an embodiment, the indicator can include a screen such as an LED screen or and LCD display. The screen can be configured to provide a simple indication of the magnitude and direction to a preferred measurement site, indication of current position at a preferred measurements site, indication that an electrical potential measurement has been successfully captured, etc. The simple indication can include alpha numeric indicators (e.g., text or numeric codes), a simple anatomical graphic indicating the position of the glove 110 or preferred measurement site, or symbolic representations of direction or distance to a preferred measurement site (e.g., an arrow of varying direction or length depending on distance and position).

In an embodiment, the at least one indicator 146 can include one or more tactile indicators. Tactile indicators (e.g., tactile indication devices) can include a tightening mechanism and associated tightening members (e.g., ratchet belt or inflation means), one or more vibrating elements, a plurality of selectively deployable nodes (e.g., selectively drivable blunt needles or buttons), or any other device configured to provide a tactile indication to the user, such as to the skin of the user. The one or more tactile indicators can be configured to operate similar or identical to any of the audio or visual indicators disclosed above. In an embodiment, any of the indicators (e.g., audio, visual, or tactile) can be used similarly or identically to any of the other indicators disclosed above. For example, the tactile indicators can be configured to pulse or vibrate at a slower or faster pace depending on the proximity of one or more portions of the glove 110 to the preferred measurement site.

In an embodiment, at least one indicator 146 can be associated with or positioned proximate to a corresponding portion of the glove 110 such that indication can be provided for substantially only that portion of the glove 110. In an embodiment, one or more indicators 146 can be positioned adjacent to each position sensor 120, electrode 130, or portion of the glove 110 (e.g., on each finger sheath 111-115 and palm 116) to provide an indication of the proximity of each position sensor 120, electrode 130, or portion of the glove 110 to one or more preferred measurement sites. For example, the processor 142 can be configured to direct or indicate instructions through the array of lights to selectively guide the user to one or more preferred measurement sites, such as by indicating which portions of the glove 110 are or are not near the preferred measurement site by varying one or more of the number of lights that are illuminated, intensity, or strobe duration to correspond with the proximity to the preferred measurement site. The user can take the indicated cues to move the glove 110 to a position wherein more or all of the indicators indicate the glove 110 or one or more components thereon are at the preferred measurement site. In such embodiments, the controller 140 via the one or more indicators 146 can selectively steer or direct (at the direction of the processor) the user to the preferred measurements sites.

Any of the indicators 146 herein can be configured in any manner described herein with respect to any other indicator 146. For example, one or more tactile indicators can be configured to provide a first tactile indication responsive to the controller 140 determining the electrode 130 is not near a preferred measurement site and a second, different tactile indication when the controller 140 determines that the electrode is near the preferred measurement site. Further, the indicators 146 herein can be configured to indicate a distance to a preferred measurement site or amount of motion require for the glove 110 to arrive thereat, such as by providing different intensities or strobes of the respective indications. For example, a slower strobe can indicate that a position sensor 120 is farther from a preferred measurement site than a faster strobe, and that the user must move the glove 110 farther than when the strobe is faster. In an embodiment, a lower pitched or lower volume audio indication can be used to indicate that the glove 110 or a portion thereof is farther from a preferred measurement site than if the audio indication was at a higher pitch or higher volume, such that the user will understand that the at least one glove 110 must be moved farther than if the audio indication had a higher pitch or volume. The controller 140 can be configured to indicate or cause the indicators to indicate a required or desired motion of the glove or a portion thereof relative to the preferred measurement site, such as by indicating one or more of the amount of motion or the direction of motion required to reach the preferred measurement site. In such a manner the controller 140 and at least one indicator can be configured to selectively steer the user to a preferred measurement site. The controller 140 can be configured to provide direction to the at least one indicator 146 to indicate any of the foregoing.

In an embodiment, the controller 140 via the at least one indicator 146 can be configured to indicate to a user that a satisfactory or complete set of measurements have been collected, such as from the at least one electrode 130 or the at least one position sensor 120. In an embodiment, the controller 140 can include program instructions configured to direct the indicator 146 to indicate completion of collecting electrical potential measurements based on one or more of a stored measurement duration, one or more measurement characteristics (e.g., collection of a specified number of QRS peaks in an EKG), a specific time of inactivity (e.g., static position) of the glove 110, etc.

In an embodiment, one or more systems 100 can be configured to communicate with each other to provide independent (e.g., simultaneous) measurements at different positions on the subject. In such embodiments, the controllers 140 thereof can transmit measurements to the other or to a remote device (e.g., a desktop computer) to be output as EKG data.

In an embodiment, more than one glove 110 can be associated with a single controller 140, such as two gloves 110 acting as different electrodes (e.g., a first glove 110 as a positive electrode and a second glove 110 acting as a negative electrode). In such embodiments, the controller 140 can be located on one glove 110 or remotely from each glove 110. In such embodiments, each glove 110 can include a communications relay configured to communicate (e.g., receive and transmit data) with the controller 140 either wirelessly or by wired connection. In such embodiments, each glove 110 can include at least one electrode 130 and at least one position sensor 120 as described above. Each of the at least one electrode 130 and position sensor 120 on each glove can communicate sensed data with the single controller 140 in a similar or identical way as those disclosed above. The controller 140 can communicate with each glove 110 or a portion thereof (e.g., individual sensors or indicators) in a similar or identical way as those disclosed above.

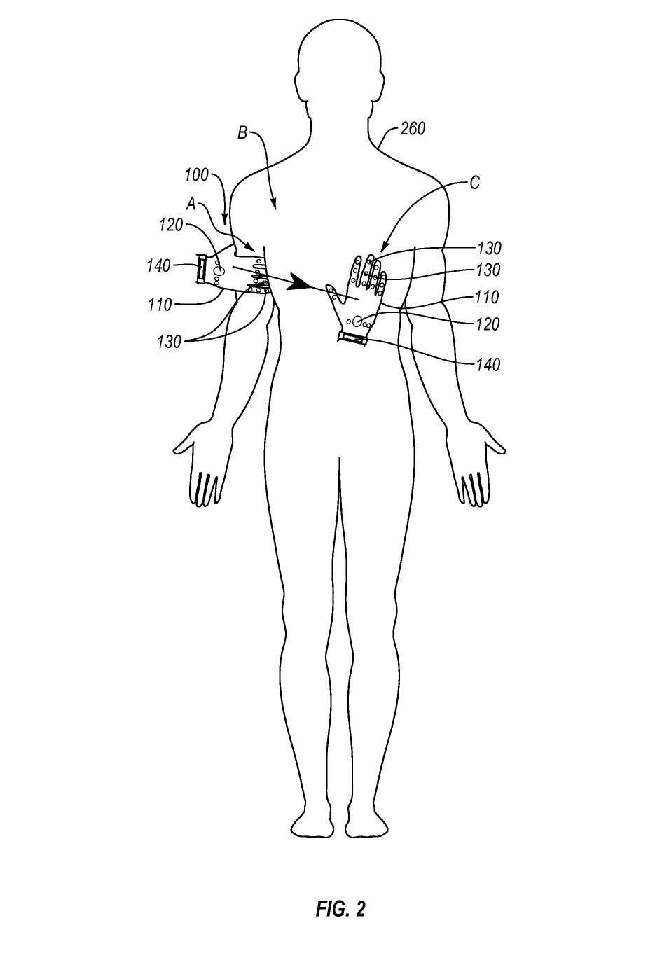

FIG. 2 is a schematic diagram of the electrocardiogram system 100 in use, according to an embodiment. In an embodiment, the glove 110 can collect electrical potential measurements from one or more locations on a subject for later compilation and analysis to determine the electrical characteristics of a subject's heart using the controller 140. The electrocardiogram system 100 can include at least one glove 110 and at least one controller 140, as disclosed above. The at least one glove 110 can be positioned on any portion of a subject 260. The at least one glove 110 can be positioned at least proximate to a lead position on the subject 260. The one or more position sensors 120 on the glove 110 can detect data related to the position of the glove 110 or one or more portions thereof and transmit the position data to the controller 140. The controller 140 can process the position data (e.g., compare the position data to an anatomical atlas or a look-up table) to determine the proximity of the glove 110 or a portion thereof to a preferred measurement site (e.g., lead location). The controller 140 can be configured to direct at least one indicator 146 to indicate the relative position of the glove 110 or a portion thereof to the preferred measurement site (e.g., a closest lead location or a specific lead location) on the subject.

In an embodiment, the at least one glove 110 can be positioned proximate to the upper arm of the subject 260 at position A. The at least one position sensor 120 located in the palm of the glove 110 can detect position data associated with the position A and transmit the same to the controller 140. The at least one position sensor 120 can include one or more of any of the position sensors 120 disclosed herein. The controller 140 can be configured to receive the position data from the at least one position sensor 120 and process the position data to determine positional characteristics (e.g., position or orientation) of the glove 110 or one or more portions thereof (e.g., the electrodes 130 in the fingers). Responsive to determining the positional characteristics, the controller 140 can be configured to determine the proximity of one or more portions of the glove 110 to the preferred measurement site adjacent thereto, such as at position B (e.g., on the upper right portion of the torso) or remote therefrom at position C (e.g., on the mid-left portion of the torso). The controller 140 can be configured to indicate one or both of the direction of or amount of movement necessary to arrive at the specified preferred measurement site or tag the electrical potential data captured thereat with the corresponding positional data. For example, the controller 140 can be configured to direct the one or more indicators (not shown) to indicate that position C is a specific direction and distance from position A. The user (e.g., medical professional) can selectively move the glove 110 to position C based on the indications or can move to any other desired lead location (e.g., position B). Once at the preferred measurement site the controller 140 can be configured to direct the indicators to indicate that the glove 110 or a portion thereof is at a preferred measurement site. Responsive to determining that the glove 110 or a portion thereof is at the preferred measurement site, the controller 140 can direct the at least one electrode 130 to collect electrical potential measurements, or can begin storing data from at least one electrode 130 (e.g., such as when the at least one electrode continuously collects data). In such embodiments, the controller 140 can be configured to direct the user to collect data (e.g., positional data or electrical potential measurements) from one or more preferred measurements sites (e.g., one or more of 10 electrode positions for a 12-lead EKG, a site of a previous measurement so as to permit historical comparisons, or the like). For example, the controller 140 can be configured to direct (e.g., store instructions thereon) the user to measure electrical potential measurements from one or more specific leads or in a specific order, such as from point C then from point B. The collective electrical potential measurements at any of the various points can be transmitted, stored, analyzed, or output from the controller 140 or a remote device (not shown) to determine the electrical characteristics of the subject's heart.

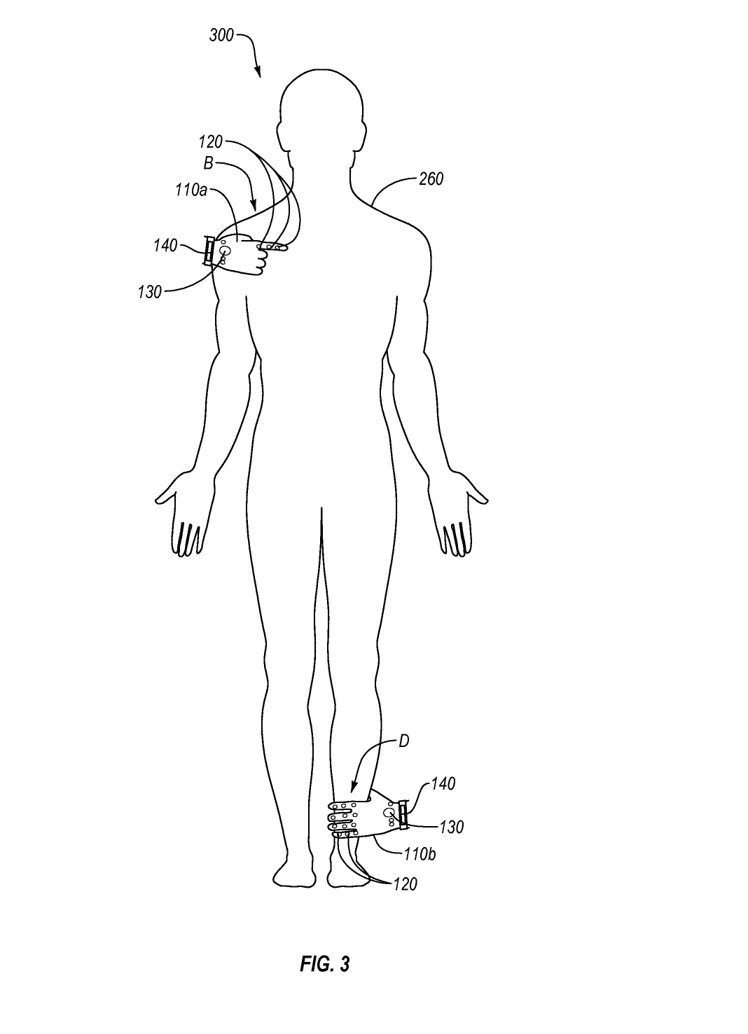

FIG. 3 is a schematic diagram of an electrocardiogram system 300 in use, according to an embodiment. In an embodiment, it may be desirable to measure the voltage at both ends of a specific lead in one instance. The system 300 can include more than one glove 110. More than one glove 110 can be worn by a single user or simultaneously by more than one user. For example, the system 300 can include a first glove 110a and a second glove 110b, which can be similar or identical to glove 110 disclosed herein, each being worn by a different user. In an embodiment, the gloves 110a and 110b can be identical to each other or different. The gloves 110a and 110b can include sensors in any configuration disclosed herein. For example, in an embodiment, the gloves 110a and 110b can include a plurality of position sensors 120 in the finger sheaths and an electrode 130 in the palm thereof. In an embodiment, the system 300 can include one or more controllers 140 disposed in one or more of the gloves 110a and 110b or remote from the one or more gloves 110a or 110b.

The one or more gloves 110a and 110b can be positioned on a subject 260, such as proximate to one or more electrode positions and on the subject 260. For example, the glove 110a can be positioned proximate to the first electrode position B and the glove 110b can be positioned proximate to a second electrode position D. The respective position sensors 120 of each of gloves 110a and 110b can be positioned in any portion thereof, such as in the finger sheaths as depicted. The respective position sensors 120 of each of gloves 110a and 110b can detect position data related to the position of the respective glove and transmit the position data to the respective controller 140 of each glove 110a or 110b. The controller 140 can determine the position of the glove 110a or 110b and indicate if movement is necessary or tag the electrical potential data captured thereat with the positional data corresponding thereto. The controllers 140 can be configured to communicate with each other to determine when each of the gloves 110a and 110b is at the selected preferred measurement site. Responsive to determining that each glove 110a and 110b is at a respective preferred measurement site, the controller 140 can be configured (e.g., include program instructions stored therein) to cause the one or more electrodes 130 in a respective glove 110a or 110b to capture electrical potential measurements or to tag the captured electrical potential measurements with the corresponding positional data indicating that the electrical potential measurements were taken at a preferred measurement site. In such embodiments, an EKG lead can be measured at once (as compared to additive measurements from a single glove at the same preferred measurement sites at different times) using substantially simultaneous measurements from the separate gloves 110a and 110b. In an embodiment, the electrical potential measurements need not be simultaneous and can be taken at different times by the different gloves 110a and 110b. In such embodiments, the electrical potential measurements can be correlated to each other at a later point in time using the controller 140 or a remote device (FIG. 5) operably coupled thereto.

In an embodiment, it may be desirable to collect one or more electrical potential measurements at one or more discrete sites on the subject 260 with more precision. In such embodiments, it may be desirable to use only a portion of the glove 110a, such as the electrodes 130 in only one finger, to capture electrical potential measurements at a discrete site. In such embodiments, the controller 140 can be programmed to deactivate one or more of the sensors in the portions of the glove 110a not being used, such that false readings are not collected. In an embodiment, only one or more portions of the glove 110a can be selectively activated or deactivated to take discrete (e.g., more precise) measurements. In an embodiment, the controller 140 can be configured to direct the indicators (not shown) to indicate which portions of the glove 110a are activated or deactivated.

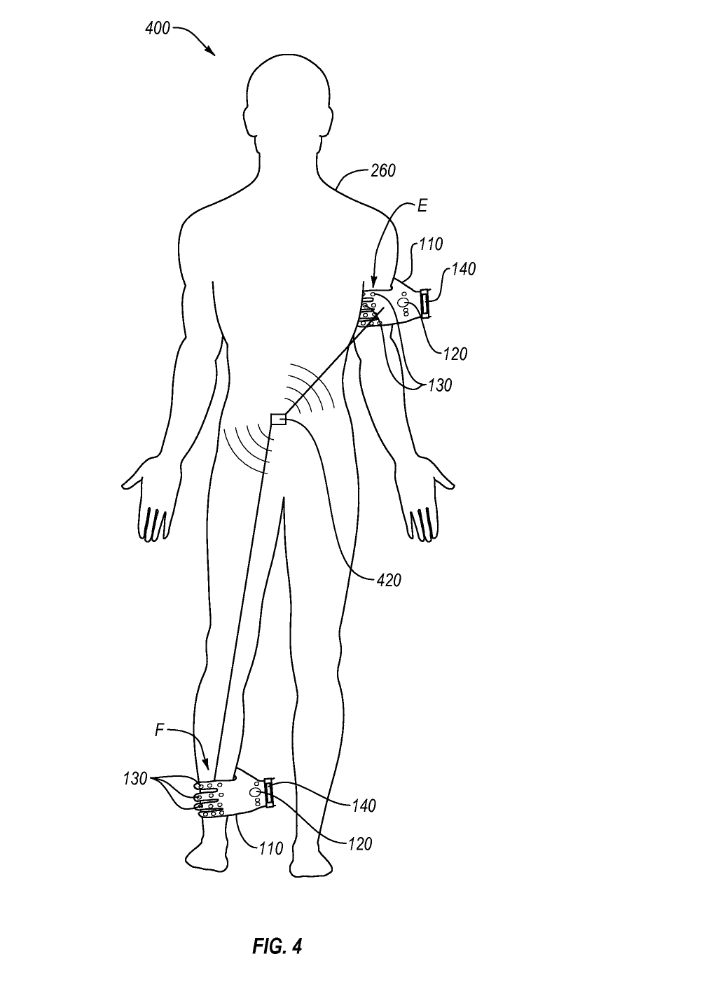

FIG. 4 is a schematic diagram of an electrocardiogram system 400 in use, according to an embodiment. The electrocardiogram system 400 can be similar or identical to the electrocardiogram system 100 disclosed above. For example, the electrocardiogram system 400 can include at least one glove 110 or controller 140 as described above. The electrocardiogram system 400 can include one or more position sensors 420 separate from the at least one glove 110. The one or more position sensors 420 can include one or more position sensors configured to detect the location of one or more objects remote therefrom. For example, the one or more position sensors 420 can include a micro-impulse radar or camera positioned on the subject, such as at a fixed point. As shown, the position sensor 420 can be positioned on the subject 260 at a central point such as the abdomen. The position sensor 420 can be configured to detect the relative positional relationship between the sensor 420 and the one or more gloves 110, or the one or more gloves 110 and the subject 260. In an embodiment, the one or more gloves 110 can include substantially only electrodes 130 and the position sensor 420 can detect and determine the position of the gloves 110 or one or more portions thereof, with respect to the subject 260. In an embodiment (not shown), the one or more gloves 110 the electrocardiogram system 400 can include position sensors 120 as described herein in addition to the position sensor 420. The positional data from the position sensor 420 or 120 can be transmitted or otherwise communicated to the controller 140. The controller 140 can include program instructions (e.g., anatomical atlas) including the relative positional relationship of the one or more position sensors 420 to the subject 260 or anatomical features of the subject, including the preferred measurement sites. The controller 140 can be configured to correlate the positional data from the position sensors 420 or 120 with the program instructions to determine the relative position of the one or more gloves 110 or one or more portions thereof.

The controller 140 can be configured to cause the glove 110 to provide one or more indications to a user as described herein. For example, the controller 140 can include program instructions stored therein effective to cause the one or more electrodes 130 to collect electrical potential measurements, tag the electrical potential measurements with corresponding positional data, discard or disregard electrical potential measurements (e.g., when the positional measurements indicate that the glove 110, electrode 130, or position sensor 120 is not at a preferred measurement site), or combinations of any of the foregoing, responsive to the determined relative position of the one or more gloves 110 or one or more portions thereof (e.g., fingers, sensors, etc.).

In an embodiment, the controller 140 can be positioned on the glove 110, on the position sensor 420 (not shown), or remote from the glove 110 and position sensor 420. In such embodiments, the position data from the position sensor 420 or the gloves 110 can be communicated thereto, such as by a wireless connections therebetween.

FIG. 5 is a schematic diagram of an electrocardiogram system 500 according to an embodiment. The system 500 can include a glove 510 similar or identical to the glove 110 disclosed herein. The system 500 can include one or more position sensors 520 similar or identical to the position sensors 120 disclosed herein. The system 500 can include one or more electrodes 530 similar or identical to the electrodes 130 disclosed herein. The system 500 can include one or more indicators 546 similar or identical to the indicators 146 disclosed herein. The one or more indicators 546 can be positioned on one or more of the glove 510 or a controller 540.

The system 500 can include the controller 540, which can be similar or identical to the controller 140 disclosed herein. The controller 540 can include control electrical circuitry 541. The control electrical circuitry 541 can be configured to communicate with and control one or more portions of the system 500 (e.g., the electrodes 530, position sensors 520, etc.). The control electrical circuitry 541 can include a processor 542 similar or identical to the processor 142 disclosed herein, which includes processing electrical circuitry. The control electrical circuitry 541 can include memory 544 similar or identical to the memory 144 disclosed herein, which can include memory electrical circuitry. The controller 540 can include a communications relay 548. The memory 544 can be operably coupled to the processor 542 such that the processor 542 can store and access information (e.g., look-up tables, anatomical atlases, program instructions, etc.) thereon. The communications relay 548 can be configured to facilitate communication between the one or more electrodes 530, the one or more position sensors 520, or a remote device 570 and the control electrical circuitry 541. In an embodiment, the communications relay 548 can be configured to communicate with a second communications relay of a second system 500 (e.g., one or more additional gloves 510 or controllers 540). The communications relay 548 can be operably coupled to one or more of the controller 540 (e.g., control electrical circuitry 541 or a portion thereof, such as the processor or the memory), the one or more position sensors 520, the one or more electrodes 530, or the remote device 570 (e.g., a computer, tablet, mobile phone, etc.), such as by direct electrical connection or wireless connection.

Each of the one or more position sensors 520, the one or more electrodes 530, and the one or more indicators 546 can be powered by a power source (not shown) operably coupled thereto. The power source can include one or more of at least one battery, an electrical connection (e.g., plug), an energy harvesting device (e.g., thermoelectric device, a kinetic energy harvesting device, a photoelectric power device, etc.), or any other suitable power source. in an embodiment, each of the one or more position sensors 520, the one or more electrodes 530, and the one or more indicators 546 can be operably coupled to (e.g., wired to or wirelessly coupled with) the same power source or different power sources, respectively. In an embodiment, each of the one or more position sensors 520, the one or more electrodes 530, and the one or more indicators 546 can be hard wired to a battery. In an embodiment, the one or more position sensors 520, the one or more electrodes 530, and the one or more indicators 546 can each have an individual power source (e.g., a battery, a wireless power supply, an energy harvesting device, etc.) associated therewith. In an embodiment, each position sensor 520 or electrode 530 in a region of the glove 510 (e.g., finger sheath or palm) can be operably coupled to a power source associated therewith, such as all of the sensors in a finger sheath being operably coupled to a power supply therein. In an embodiment, at least one of the one or more position sensors 520, the one or more electrodes 530, and the one or more indicators 546, can be operably coupled to a power source 550 associated with (e.g., disposed in, on, or operably coupled to) the controller 540, such as by one or more operable connections. The operable connections 526 can include wireless connections or hard wired connections (e.g., electrical connections 125) or wireless connections.

In operation, the one or more position sensors 520 or one or more electrodes 530 can transmit sensed data to the controller 540 via the communications relay 548. The sensed data can be routed to the processor 542 or the memory 544. The communications relay 548 can relay program information to at least one of the one or more position sensors 520 or one or more electrodes 530. For example, the memory 544 can include an operational program stored thereon. The operational program can be configured to direct the one or more electrodes 530 not to collect electrical potential measurements until the processor 542 determines that the positional measurements indicate that the glove 510 or a portion thereof (e.g., one or more electrodes 530) is at a preferred measurement site. In an embodiment, the memory 544 can include an operational program stored thereon, the operational program configured to instruct the processor 542 to ignore all incoming electrical potential measurements until the processor 542 determines that the positional measurements indicate that the glove 510 or a portion thereof is at a preferred measurement site.

The processor 542 can be configured to determine if one or more of the position sensors 520, one or more electrodes 530 adjacent thereto, or glove 510 is at or adjacent to a preferred measurement site, or is not near a preferred measurement site, in any manner disclosed herein. In an embodiment, the processor 542 can be configured to compare and correlate the sensed position data (e.g., acoustic image data, visual image data, audio data, motion data, etc.) to one or more look-up tables, anatomical atlases, or other data stored in the memory 544 to determine if the one or more of the position sensors 520, one or more electrodes 530 adjacent thereto, or glove 510 is at a preferred measurement site. Responsive to determining that a correlation has been established, such as by a match to an anatomical atlas, the processor can be configured to direct the indicators 546 to provide an indication to the user that the glove 510 or a component thereof is at the preferred measurement site. In an embodiment, responsive to the processor 542 determining that the glove 510 or a portion thereof is not at a preferred measurement site, the processor 542 can direct the indicators 546 to indicate that the glove 510 or a portion thereof is not at a preferred measurement site, such as with an indication differing from the indication that the glove 510 is at the preferred measurement site (e.g., different tone, different lights, different strobe pattern, different tactile indication, etc.). In an embodiment, the memory 544 can include an operational program carried out by the processor 542, the operational program configured to direct the one or more indicators 546 to steer the user to one or more preferred measurement sites using indications as disclosed herein with respect to indicators 146.

In an embodiment, the processor 542 can be configured to compare one or more electrical potential measurements with one or more stored (e.g., stored in the memory 544) electrical potential measurements, such as stored electrical potential measurements of the electrical potential at a preferred measurement site, electrical potential measurements of an average healthy member of a demographic (e.g., ranges of electrical potential measurements of a healthy subject), or previously stored electrical potential measurements of the subject. Via the comparison, the processor 542 can be configured to determine (e.g., such as by a best fit analysis) if the electrical potential measurements were taken at a preferred measurement site, determine an EKG based on multiple leads on the same subject, or determine if the subject's measurements deviate from the measurements of healthy individuals or the individual's past measurements. In an embodiment, the processor 542 can be configured to correlate, an optionally tag, one or more electrical potential measurements with one or more positional measurements over a selected amount of time to provide a time-sequenced measured electrical potential at specific position or group of positions on the subject. The processor 542 can be configured to compare the time-sequenced measured electrical potential at the specific location on a subject to one or more stored standard electrical potential measurements of one or more leads, such as one or more time-sequenced electrical potential measurements for an average subject in a demographic or one or more stored time-sequenced electrical potential measurements of the subject being examined. The processor 542 can be configured to store any of the above measurements or determined data in the memory 544 or to output the same to the remote device 570 (e.g., special purpose computer associated therewith).

The remote device 570 can be configured to display, store, or further process the information from one or more the at least one electrode 530, the position sensors 520, processor 542, or the memory 544. The remote device 570 can be configured to transmit instructions, data, or other information to the controller 540, such as via the communications relay 548. For example, the communications relay 548 can include a wireless receiver configured to receive program instructions, data associated with the preferred measurement site, the data associated with the preferred measurement site including at least one of one or more measurement sites measured or electrical potential measurements thereat, location of the one or more measurement sites, or data used to describe each site of the one or more measurement sites, such as from one or more of the electrodes 530, the position sensors 520, another system 500, or the remote device 570.

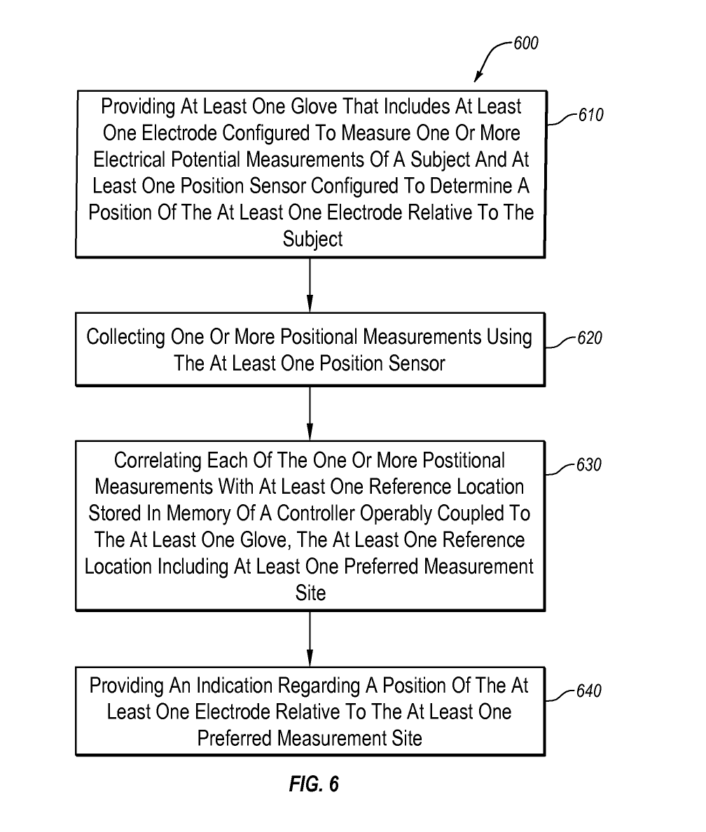

FIG. 6 is a flow chart of an embodiment of a method 600 of determining proper placement of sensors of at least one glove for determining electrical characteristics of a cardiovascular system of a subject. The method 600 can include an act 610 of providing the at least one glove that includes at least one electrode configured to measure one or more electrical potential measurements of a subject and at least one position sensor configured to determine a position of the at least one electrode relative to the subject. The method 600 can include an act 620 of collecting one or more positional measurements. The method 600 can include an act 630 of correlating each of the one or more positional measurements with at least one reference location stored in memory of a controller operably coupled to that at least one glove or one or more portions thereof, the at least one reference location including at least one preferred measurement site. The method 600 can include an act 640 of providing an indication regarding a position of the at least one electrode.

The act 610 of providing the at least one glove that includes at least one electrode configured to measure one or more electrical potential measurements of a subject and at least one position sensor configured to determine a position of the at least one electrode relative to the subject can include using any glove or component associated therewith disclosed herein. For example, the at least one glove, at least one position sensor, or at least one electrode can be similar or identical to any glove, electrode, or position sensor disclosed herein, respectively.

In an embodiment, the at least one position sensor can include at least one optical sensor configured to capture at least one image of the subject and relay the same to the controller. The at least one optical sensor can include one or more of a camera, a micro-camera, a topography sensor, or an infrared sensor. In an embodiment, the at least one position sensor can include at least one audio sensor configured to capture at audio information or data of the subject and relay the same to the controller. The at least one audio sensor can include a microphone, an acoustic sensor (e.g., a ultrasound Doppler sensor), or a sound detector. In an embodiment, the at least one position sensor can include at least one acoustic sensor configured to capture at least acoustic one image of the subject and relay the same to the controller. The at least one audio sensor can include an ultrasound sensor, such as an ultrasound Doppler sensor. In an embodiment, the at least one position sensor can include a topography sensor configured to capture one or more sets of topographical data of the subject and relay same to the controller. The topography sensor can include a video camera or infrared camera configured to capture one or more images of the skin of the subject. In an embodiment, the at least one position sensor can include at least one accelerometer configured to determine one or more changes in position of the position sensor from a first time to a second time and transmit the same to the controller. In an embodiment, the at least one position sensor can include at least one contact sensor configured to configured to detect if the at least one glove or a portion thereof is in sufficient contact with the subject to sense selected data and transmit the same to the controller. The at least one contact sensor can include one or more of one of a pressure sensor, a stress sensor, an ultrasonic transducer, an electrical resistance sensor, or at least one audio sensor configured to sense heart beat or blood flow of the subject.

In an embodiment, the at least one glove can include at least one electrode, such as a plurality of electrodes as disclosed herein. In an embodiment, each of the at least one electrodes can be associated with an individual position sensor of a plurality of position sensors. In an embodiment, each finger of a glove can include at least one position sensor and at least one electrode therein or thereon. In an embodiment, the palm of the glove can include at least one position sensor and at least one electrode therein or thereon.

In an embodiment, the at least one glove can include one or more indicators associated therewith (e.g., affixed thereto). The at least one indicator can include one or more of any indicator disclosed herein (e.g., visual indicator, audio indicator, tactile indicator, etc.). In an embodiment, at least one of the at least one indicators can be associated with each electrode, each position sensor, or each finger on the glove, to provide indication of the position thereof. In an embodiment, the at least one indicator can be configured to provide an indication that the positional measurements or at least a portion of the at least one glove is at or near a preferred measurement site, or not at or near a preferred measurement site. In an embodiment, the at least one indicator can be configured to provide an indication of the direction or amount of motion necessary to position at least a portion of the at least one glove at or near the preferred measurement site.

The method 600 can include the act 620 of collecting one or more positional measurements using the at least one glove. For example, collecting one or more positional measurements can include collecting one or more positional measurements using at least one position sensor. In an embodiment, collecting one or more positional measurements using at least one position sensor can include using a plurality of position sensors. The plurality of position sensors can include a plurality of the same type of position sensor or one or more different sensors, such as any position sensor disclosed herein. Collecting one or more positional measurements can include collecting any of the positional measurements disclosed herein, such as motion data, optical data, one or more images, acoustic data, audio data, contact data, etc.

In an embodiment, collecting one or more positional measurements using at least one position sensor can include using at least one optical sensor and capturing at least one image of the subject. In an embodiment, collecting one or more positional measurements using at least one acoustic sensor and collecting one or more positional measurements includes capturing at least acoustic one image of the subject. In an embodiment, collecting one or more positional measurements includes using a topography sensor and capturing one or more sets of topographical data of the subject, such as one or more images or infrared images. In an embodiment, collecting one or more positional measurements can include using an accelerometer and capturing one or more changes in position of the accelerometer, such as direction, velocity, orientation, or acceleration.

The method 600 can include the act 630 of correlating each of the one or more positional measurements with at least one reference location. In an embodiment, correlating each of the one or more positional measurements with at least one reference location can be carried out by the control electrical circuitry, specifically the processor, of the controller associated with the at least one glove. In an embodiment, the at least one reference location can be stored in memory of the controller, which can be accessed by the processor. The at least one reference location can include at least one preferred measurement site, such as a plurality of preferred measurement sites. The preferred measurement sites can include the lead locations (e.g., lead locations for an average subject or the subject being examined) for each electrode for an accurate EKG. Correlating each of the one or more positional measurements with at least one reference location can include comparing each of the one or more positional measurements to a look-up table, anatomical atlas, or other data including the at least one reference location stored in the memory. In an embodiment, correlating each of the one or more positional measurements with at least one reference location can include determining if the one or more positional measurements are or are not near (e.g., are or are not within a distance selected to allow effective and reliable electrical potential measurements to be collected) the at least one reference location. In an embodiment, correlating each of the one or more positional measurements with at least one reference location can include determining if the at least one reference location is at or near at least one preferred measurement site. In an embodiment, determining if the at least one reference location is at or near at least one preferred measurement site can include comparing each of the one or more positional measurements to a look-up table, anatomical atlas, or other data including the at least one reference location stored in the memory.

In an embodiment, correlating each of the one or more positional measurements with at least one reference location can include correlating at least one image with the at least one reference location stored in the memory, such as a reference location image. In an embodiment, correlating each of the one or more positional measurements with at least one reference location can include determining if the at least glove or a portion thereof (e.g., at least one electrode) is within a distance stored in the memory as specified for effective use of the at least one glove or a portion thereof. In an embodiment, the processor can be configured to compare and provide a best fit analysis (e.g., including scale, orientation, and position) using the at least one image and an anatomical atlas to determine the relative location of the at least one image and position of the at least one glove or a portion thereof on the subject.

In an embodiment, correlating each of the one or more positional measurements with at least one reference location stored in memory can include correlating at least one acoustic image with the at least one reference location stored in the memory, such as in and acoustic image (e.g., anatomical atlas image) having the reference location therein. In an embodiment, correlating each of the one or more positional measurements with at least one reference location stored in memory can further include determining if the at least one glove or a portion thereof is within a distance stored in the memory as specified for effective use of the same. In an embodiment, the processor can be configured to compare and provide a best fit analysis using the at least one acoustic image and an anatomical atlas to determine the relative location of the at least one image and position of the at least one glove or a portion thereof on the subject.

In an embodiment, correlating each of the one or more positional measurements with at least one reference location can include correlating one or more sets of topographical data of the subject with the at least one reference location. The at least one reference location can be stored in the memory. In an embodiment, the processor can be configured to compare and provide a best fit analysis using the at least topographical data (e.g., image or IR image) and an anatomical atlas including topographical data to determine the relative location of the topographical data and position of the at least one glove or a portion thereof on the subject. In an embodiment, correlating each of the one or more positional measurements with at least one reference location can include determining if the at least glove or a portion thereof is within a distance stored in the memory as specified for effective use of the same. In an embodiment, the processor can be configured to compare and provide a best fit analysis using the topographical data and an anatomical atlas or look-up table to determine the relative location of the at least one image and position of the at least one glove or a portion thereof on the subject.

In an embodiment, correlating each of the one or more positional measurements with at least one reference location can include correlating one or more changes in position of a position sensor with the at least one reference location. In an embodiment, the processor can be configured to determine from the relative motion of one or more position sensors, the position of the same at any point in time. For example, when an accelerometer is used, the accelerometer data can indicate the orientation of the glove or a portion thereof, the direction of movement of the glove or a portion thereof, or the velocity or acceleration of the glove or a portion thereof. The accelerometer data can be used to determine the position of the glove at any point in time, such as by calculating a current position by determining the position of the at least glove or a portion thereof based on a known start position (e.g., a reference position) and the one or more changes in position captured by the accelerometer. The position sensor data and at least one reference location can be stored in the memory. In an embodiment, correlating each of the one or more positional measurements with at least one reference location can include determining if the at least glove or a portion thereof is within a distance (stored in the memory) of the preferred measurement site specified for effective use of the glove (e.g., detection of electrical potential of a specific lead). In an embodiment, the processor can be configured to compare the position data and an anatomical atlas or look-up table to determine the position of the at least one glove or a portion thereof on the subject.

The method 600 can include the act 640 of providing an indication regarding a position of the at least one glove or a portion thereof such as one or more electrodes or position sensors. In an embodiment, providing an indication regarding the position of the at least one electrode can include providing the indication of the at least one electrode relative to the at least one preferred measurement site. The at least one preferred measurement site can be the closest preferred measurement site or a selected preferred measurement site. In an embodiment, providing an indication regarding the position of the at least one electrode can include one or more of providing an audio indication, providing a tactile indication, or providing a visual indication that at least a portion of the at least one glove (e.g., at least one electrode) is at or near (e.g., within a distance wherein acceptably accurate electrical potential measurements can be collected) a preferred measurement site. The audio indication, tactile indication, or visual indication can include any of those audio (e.g., one or more tones), tactile (e.g., one or more vibrations), or visual (e.g., one or more lights) indications disclosed above, respectively.

In an embodiment, providing an audio indication can include one or more tones, such as any of the tones disclosed herein. The one or more tones can chime at different intervals depending on the relative distance of the glove from the preferred measurement site. For example, the controller can be configured to direct the audio indicator to chime between or at shorter intervals (e.g., faster) when the processor determines that the glove or a portion thereof (e.g., at least one electrode or at least one position sensor) is as at or near the preferred measurement site and the indicator to chime between or at longer intervals (e.g., slower) when the glove or a portion thereof is farther from the at least one preferred measurement site. In an embodiment, the one or more tones can include varying volume depending on the determined distance of the at least one glove or a portion thereof from a preferred measurement site. For example, the controller can be configured to direct the at least one indicator to chime at a higher or lower volume to indicate a relative position of the at least one glove or a portion thereof. For example, the audio indicator and controller can be configured to provide an increase in the volume of the tone when at the glove or a portion thereof (e.g., position sensor or electrode) is closer to the at least one preferred measurement site and decrease in volume when the same is farther from the at least one preferred measurement site. In an embodiment, the one or more tones can include varying pitch or frequency. For example, the controller can be configured to direct the at least one indicator to emit one or more tones at a higher or lower tone to indicate a relative position of the at least one glove or a portion thereof. In an embodiment, the one or more tones can include a first higher tone when the at least one electrode is closer to the at least one preferred measurement site and a second lower tone when the at least one electrode is farther from the at least one preferred measurement site.

In an embodiment, providing a visual indication can include providing one or more lights emitted from the at least one indicator, such as any of the lights disclosed herein. The one or more lights can emit (e.g., flash) at different intervals depending on the relative distance of the glove from the preferred measurement site. For example, the controller can be configured to direct the lights to emit between or at shorter intervals when the processor determines that the glove or a portion thereof is as at or near the preferred measurement site and direct the lights to emit between or at longer intervals when the glove or a portion thereof is farther from the at least one preferred measurement site. In an embodiment, the one or more lights can include varying intensity or color depending on the determined distance of the at least one glove or a portion thereof from a preferred measurement site. For example, the controller can be configured to direct the at least one indicator to emit a brighter light or dimmer to indicate a relative position of the at least one glove or a portion thereof. For example, the visual indicator and controller can be configured to provide an increase in intensity of emitted light when at the glove or a portion thereof is closer to the at least one preferred measurement site and decrease in intensity when the same is farther from the at least one preferred measurement site. In an embodiment, the controller can be configured to direct the at least one indicator to emit a first color of light or and at least a second color of light to indicate a relative position of the at least one glove or a portion thereof. For example, the controller and visual indicator can cause the lights to flash in a first color when the at least one electrode is closer to the at least one preferred measurement site and emit or change color to a second color when the at least one electrode is farther from the at least one preferred measurement site. In an embodiment, the controller can be configured to direct the at least one indicator to cause one or more lights of a plurality of lights to light up or cease emitting light to indicate a relative position of the at least one glove or a portion thereof. In an embodiment, the one or more lights can include more lights when the at least one electrode is closer to the at least one preferred measurement site and fewer lights when the at least one electrode is farther from the at least one preferred measurement site.

In an embodiment, providing a tactile indication can include providing one or more of any tactile indication disclosed herein, such as one or more of pressure, vibration, or touch. The tactile indications can be provided by one or more of any suitable tactile indication device disclosed herein, such as a tightening mechanism, a vibrating element, or a plurality of selectively deployable nodes. Any of the tactile indications can be provided in a similar or identical manner as the audio of visual indicators disclosed above to indicate the relative position of at least a portion of the at least one glove to the preferred measurements site. For example, in an embodiment, the tactile indication device includes a vibrating element and the vibrating element vibrates in shorter intervals or stronger vibrations when the at least one electrode is closer to the at least one preferred measurement site and longer intervals or weaker vibrations when the at least one electrode is farther from the at least one preferred measurement site. In an embodiment, the tactile indication device includes a tightening mechanism and the tightening mechanism tightens a portion of the at least one glove around a portion of the hand of a user, to selectively apply pressure, when the at least one electrode is closer to the at least one preferred measurement site and the tightening mechanism loosens a portion of the at least one glove around the portion of the hand of a user, to selectively release pressure thereon, when the at least one electrode is farther from the at least one preferred measurement site. In an embodiment, the tactile indication device can include a plurality of selectively deployable nodes. The selectively deployable nodes and the controller can be configured to selectively deploy the plurality of selectively deployable nodes to contact the user when the at least one electrode is closer to the at least one preferred measurement site and selectively un-deploy when the at least one electrode is farther from the at least one preferred measurement site.

In an embodiment, providing an indication regarding a position of the at least one glove or a portion thereof can include providing a plurality of indications regarding the positions of each of more than one portion of the at least one glove. Providing the plurality of indications can include using a plurality of indicators, each associated with an adjacent portion of the at least one glove (e.g., electrode). In an embodiment, providing an indication regarding a position of the at least one glove or a portion thereof can include providing individual indications that each adjacent portion of the glove (e.g., electrode or position sensor) is at or near the at least one preferred measurement site, such as with an individual adjacent one of the plurality of indicators.

In an embodiment, an indication regarding a position of the at least one glove or a portion thereof can include providing one or more indications configured to selectively steer the user to one or more preferred measurement sites. Selectively steering the user to one or more preferred measurement sites can include providing an indication that the at least one glove or a portion thereof is or is not at or near a preferred measurement site. Selectively steering the user to one or more preferred measurement sites can include providing an indication of the distance or direction that the at least one glove or a portion thereof must move in order to arrive at the preferred measurement site. Any of the indicators disclosed herein can be used to selectively steer the user to the at least one preferred measurement site. For example, lights of differing intensity or number, tones of differing volume, or tactile indications of differing intensity (e.g., more or less pressure) can be used to indication which direction or how far the user must move the glove or a portion thereof to arrive at the preferred measurement site. The controller can be configured to compare the current position of the glove or a portion thereof and to the nearest or a selected preferred measurement site; calculate the required distance and direction that the glove must be moved; and send directions to the indicators to indicate the same to the user.

In an embodiment, the method 600 can include an act of collecting one or more electrical potential measurements with the at least one electrode. Collecting one or more electrical potential measurements with the at least one electrode can include collecting a plurality of electrical potential measurements at more than one location on the subject. Collecting one or more electrical potential measurements can include capturing at least one time sequence of electrical potential measurements at one or more locations on the subject. Collecting one or more electrical potential measurements with the at least one electrode can include collecting one or more electrical potential measurements over a period of time including 1 second or more, 5 seconds or more, 10 seconds or more, about 5 seconds to about 20 minutes, about 10 seconds to about 2 minutes, about 3 seconds to about 1 minute, or about 30 seconds. Collecting one or more electrical potential measurements with the at least one electrode can include selectively collecting electrical potential measurements only at a preferred measurement site as determined by the controller. Collecting one or more electrical potential measurements with the at least one electrode can include collecting electrical potential measurements at any site wherein sufficient contact with the subject is present as determined by the controller.

The method 600 can include tagging each of the one or more electrical potential measurements with corresponding position data. For example, the processor can tag each electrical potential measurement with positional data corresponding to the position of each electrode at the time that each electrical potential measurement was taken. The processor can selectively tag each electrical potential measurement from each specific electrode with position data corresponding to a nearest position sensor. Such positional data can include an image, an acoustic image, audio data, motion data (e.g., velocity or acceleration of the at least one glove during the electrical potential measurement), or any other position data describe herein. For example, tagging each of the one or more of the electrical potential measurements with position data can include tagging each electrical potential measurement with data regarding motion of the at least one electrode during the electrical potential measurement. The tagged measurements can be stored in the memory or transmitted to a remote device.

The method 600 can include accepting or rejecting one or more electrical potential measurements if the position data indicates that the position for a corresponding electrode exceeded or met a specified threshold during the electrical potential measurement. The threshold can include a minimum or maximum distance from preferred measurement site, velocity of the glove or a portion thereof during the electrical potential measurement, level of contact (e.g., amount of pressure) with the subject, or any other criteria which can adversely affect the effective measurement of electrical potential for and EKG. The threshold levels can be stored in the memory in one or more look-up tables which can be accessed and compared by the processor. In an embodiment, the one or more electrical potential measurements can be ignored, discarded, or rejected if one or more changes in position of the position sensor during the electrical potential measurement exceeds a specified threshold. In an embodiment, the one or more electrical potential measurements can be ignored, discarded, or rejected if the contact of the position sensor during the electrical potential measurement does not meet a specified threshold. In an embodiment, one or more electrical potential measurements can be tagged with position data or saved in the memory only if the position data indicates that the corresponding electrical potential measurements were taken when the position of the glove or a portion thereof met or did not exceed a specified threshold, such as distance from a preferred measurement site, motion of the glove or a portion thereof, level contact of the at least one glove or a portion thereof to the subject, or any other suitable criteria.

In an embodiment, the method 600 can include transmitting data associated with at least one of the one or more electrical potential measurements and one of the one or more positional measurements to a remote device, such as by wireless transmission, over a hard wired cable or fiber, or via a removable memory storage device (e.g., a disc or flash drive). In an embodiment, program instructions for any of the foregoing acts can be transmitted to the controller via a remote device by any of the transmission means disclosed herein. In an embodiment, threshold levels for any of the foregoing position data can be transmitted to and stored in the controller by any of the transmission means disclosed herein.

The reader will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. The reader will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. The reader will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In an embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the reader will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, the various embodiments described herein can be implemented, individually and/or collectively, by various types of electro-mechanical systems having a wide range of electrical components such as hardware, software, firmware, or virtually any combination thereof; and a wide range of components that may impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electro-mechanical systems include but are not limited to a variety of consumer electronics systems, as well as other systems such as motorized transport systems, factory automation systems, security systems, and communication/computing systems. Those skilled in the art will recognize that electro-mechanical as used herein is not necessarily limited to a system that has both electrical and mechanical actuation except as context may dictate otherwise.

In a general sense, the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). The subject matter described herein may be implemented in an analog or digital fashion or some combination thereof.

Additionally, as will be appreciated by one of ordinary skill in the art, principles of the present disclosure, including components, may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create a means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

In an embodiment, an electrocardiogram system disclosed herein can be integrated in such a manner that the electrocardiogram system operates as a unique system configured specifically for the function of determining a position of one or more electrodes in the electrocardiogram system, and any associated computing devices of the electrocardiogram system operate as specific use computers for purposes of the claimed system, and not general use computers. In an embodiment, at least one associated computing device of the electrocardiogram system operates as a specific use computer for purposes of the claimed system, and not general use computers. In an embodiment, at least one of the associated computing devices of the electrocardiogram system is hardwired with a specific ROM to instruct the at least one computing device. In an embodiment, one of ordinary skill in the art recognizes that the electrocardiogram system effects an improvement at least in the technological field of electrocardiogram devices and systems.

The herein described components (e.g., steps), devices, and objects and the discussion accompanying them are used as examples for the sake of conceptual clarity. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural and/or singular terms herein, the reader can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

In some instances, one or more components may be referred to herein as “configured to.” The reader will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense the convention (e.g., “a system having at least one of A, B, or C” would include but not be limited to systems that have A alone, B alone, C alone, A and B together, A and C together, B and C together, and/or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, the recited operations therein may generally be performed in any order. Examples of such alternate orderings may include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

While various aspects and embodiments have been disclosed herein, the various aspects and embodiments disclosed herein are for purposes of illustration and are not intended to be limiting, with the true scope and spirit being indicated by the following claims.

What is claimed is:

1. A system for capturing electrocardiogram readings, the system comprising:
at least one glove including,
at least one electrode configured to measure one or more electrical potential measurements of a subject; and
at least one position sensor configured to measure one or more positional measurements of the at least one electrode relative to the subject; and
a controller operably coupled to the at least one electrode and the at least one position sensor, the controller including a processor configured to determine if the at least one electrode is at or near a preferred measurement site for measuring the one or more electrical potential measurements at least partially based on the one or more positional measurements.

2. The system of claim 1, wherein the one or more electrical potential measurements include a time sequence of electrical potential measurements.

3. The system of claim 1, wherein the controller is configured to tag at least one of the one or more electrical potential measurements with data regarding motion including one or more of a positional change, a velocity, or an acceleration of the at least one electrode during the one or more electrical potential measurements.

4. The system of claim 1, wherein the controller is configured to reject the one or more electrical potential measurements if motion of the at least one electrode during the one or more electrical potential measurements exceeds a specified threshold.

5. The system of claim 1, wherein the at least one position sensor includes one or more of a topography sensor, an optical sensor, an acoustic sensor, an accelerometer, or a micro-impulse radar.

6. The system of claim 1, wherein:
the controller includes memory including preferred measurement sites or preferred measurement characteristics stored therein;
the processor is further configured to compare at least one of the one or more electrical potential measurements or the one or more positional measurements with corresponding measurements associated with the preferred measurement sites or the preferred measurement characteristics and determine if the at least one electrode is at or near a specific preferred measurement site of the preferred measurement sites; and
the at least one glove includes an indicator configured to indicate if the at least one glove is at the specific preferred measurement site responsive to a comparison by the processor of the one or more positional measurements and the specific preferred measurement site.

7. The system of claim 6, wherein the indicator includes a tactile indication device including one or more of a tightening mechanism, a vibrating element, or a plurality of selectively deployable nodes.

8. The system of claim 7, wherein the indicator includes one or more of a light affixed to a surface of the at least one glove or an audible indicator configured to produce one or more tones.

9. The system of claim 6, wherein, responsive to the one or more positional measurements, the processor is further configured to direct the indicator to produce a stimulus that indicates that the at least one electrode is not at or near the preferred measurement site and a second stimulus that indicates that the at least one electrode is at or near the preferred measurement site.

10. (canceled)

11. The system of claim 1, wherein the at least one position sensor includes at least one topography sensor configured to detect a topography of a skin of the subject.

12. The system of claim 1, wherein the at least one position sensor includes at least one optical sensor configured to capture one or more images of the subject and relay the one or more captured images to a memory in the controller.

13. The system of claim 12, wherein:
the controller includes memory having a database of reference images including biological structures capable of serving as reference points for positional determinations; and
the processor is further configured to compare the one or more captured images with one or more reference images in the database of reference images to determine a position of the at least one electrode.

14. The system of claim 13, wherein the at least one optical sensor includes at least one of a camera configured to capture images of the subject or an infrared detector configured to detect infrared images of the subject.

15. The system of claim 14, wherein the infrared detector is configured to capture images of the blood vessels of the subject and transmit the captured images to the memory.

16. The system of claim 15, wherein:
the one or more reference images include blood vessel reference images; and
the processor is further configured to compare the captured images of blood vessels of the subject to the blood vessel reference images to determine a position of the at least one electrode.

17. The system of claim 1, wherein the at least one position sensor includes at least one accelerometer configured to determine a change in position from a first time to a second time.

18. The system of claim 1, wherein the at least one position sensor includes at least one acoustic sensor configured to capture one or more acoustic images of the subject and relay the one or more acoustic images to the processor.

19. The system of claim 18, wherein the at least one acoustic sensor includes an ultrasound sensor configured to capture one or more ultrasonic images of the subject.

20. The system of claim 19, wherein:
the controller includes memory having reference images stored therein; and
the processor is further configured to compare the one or more ultrasonic images of the subject with the reference images stored in the memory to determine a position of the at least one electrode.

21. The system of claim 1, wherein the at least one position sensor includes a micro-impulse radar configured to detect one or more internal structures or one or more external structures of the subject and a position of the at least one electrode relative to the one or more internal structures or the one or more external structures.

22. (canceled)

23. The system of claim 1, wherein the at least one electrode is positioned in at least one finger of the at least one glove.

24. The system of claim 1, wherein:
the at least one electrode includes a plurality of electrodes; and
the at least one glove includes a plurality of fingers each of which includes one of the plurality of electrodes therein or thereon.

25. (canceled)

26. The system of claim 1, wherein the at least one position sensor is positioned in at least one finger of the at least one glove.

27. The system of claim 1, wherein:
the at least one position sensor includes a plurality of position sensors; and
the at least one glove includes a plurality of fingers each of which includes one of the plurality of position sensors therein or thereon.

28. The system of claim 1, wherein the at least one glove includes a palm having the at least one position sensor therein or thereon.

29. The system of claim 1, wherein:
the at least one position sensor includes a plurality of position sensors;
the at least one electrode includes a plurality of electrodes; and
the at least one glove includes a plurality of fingers each of which includes one of the plurality of position sensors therein or thereon and one of the plurality of electrodes therein or thereon.

30. (canceled)

31. (canceled)

32. (canceled)

33. The system of claim 1, wherein the at least one glove includes at least one contact sensor configured to determine if the at least one glove is in sufficient contact with the subject to sense selected data.

34. (canceled)

35. (canceled)

36. The system of claim 1, wherein:
the at least one glove includes an indicator configured to indicate if the at least one glove is at or near the preferred measurement site; and
the processor is further configured to direct the indicator to produce a stimulus that indicates that the at least one electrode is at or near the preferred measurement site or not at or near the preferred measurement site.

37. The system of claim 1, wherein the at least one glove includes an indicator configured to indicate a desired motion of the glove relative to the preferred measurement site.

38. (canceled)

39. The system of claim 1 wherein the at least one glove includes an indicator configured to indicate completion of collecting an electrical potential measurement.

40. (canceled)

41. The system of claim 1, wherein the at least one glove includes memory configured to store data associated with at least one of the one or more electrical potential measurements and one of the one or more positional measurements.

42. (canceled)

43. The system of claim 1, further including a wireless receiver configured to receive data associated with the preferred measurement site, the data associated with the preferred measurement site including at least one of one or more measurement sites measured or the one or more electrical potential measurements therefrom, location of the one or more measurement sites, or data used to describe each of the one or more measurement sites.

44. A method of determining proper placement of at least one electrode of at least one glove for determining electrical characteristics of a cardiovascular system of a subject, the method comprising:
providing the at least one glove that includes the at least one electrode configured to measure one or more electrical potential measurements of a subject and at least one position sensor configured to determine a position of the at least one electrode relative to the subject;
collecting one or more positional measurements using the at least one position sensor;
correlating each of the one or more positional measurements with at least one reference location stored in memory of a controller operably coupled to the at least one glove, the at least one reference location including at least one preferred measurement site; and
providing an indication regarding a position of the at least one electrode relative to the at least one preferred measurement site.

45. The method of claim 44, wherein:
the at least one glove includes at least one indicator configured to provide an indication that the one or more positional measurements or the at least one electrode is at or near the at least one preferred measurement site; and
providing an indication regarding a position of the at least one electrode relative to the at least one preferred measurement site includes one or more of providing an audio indication, providing a tactile indication, or providing a visual indication that the at least one electrode is at or near the at least one preferred measurement site.

46. (canceled)

47. (canceled)

48. (canceled)

49. (canceled)

50. The method of claim 45, wherein the visual indication includes one or more lights emitted from the at least one indicator.

51. The method of claim 50, wherein the one or more lights flash at shorter intervals when the at least one electrode is closer to the at least one preferred measurement site and the one or more lights flash at longer intervals when the at least one electrode is farther from the at least one preferred measurement site.

52. The method of claim 50, wherein the one or more lights increase in intensity when the at least one electrode is closer to the at least one preferred measurement site and decrease in intensity when the at least one electrode is farther from the at least one preferred measurement site.

53. The method of claim 50, wherein the one or more lights flash in a first color when the at least one electrode is closer to the at least one preferred measurement site and change color to a second color when the at least one electrode is farther from the at least one preferred measurement site.

54. The method of claim 45, wherein the at least one indicator includes a tactile indication device including one or more of a tightening mechanism, a vibrating element, or a plurality of selectively deployable nodes.

55. (canceled)

56. (canceled)

57. (canceled)

58. The method of claim 45, wherein:
the at least one indicator includes a plurality of indicators each of which is associated with an adjacent electrode; and
providing an indication regarding a position of the at least one electrode relative to the at least one preferred measurement site includes providing individual indications that each adjacent electrode is at or near the at least one preferred measurement site with an individual adjacent one of the plurality of indicators.

59. The method of claim 44, wherein:
the at least one position sensor includes an optical sensor configured to capture at least one image of the subject and relay the at least one image to the controller;
collecting one or more positional measurements includes capturing at least one image of the subject; and
correlating each of the one or more positional measurements with at least one reference location stored in memory includes,
correlating the at least one image with the at least one reference location stored in the memory; and
determining if the at least one electrode is within a distance stored in the memory as specified for effective use of the at least one electrode.

60. (canceled)

61. The method of claim 44, wherein:
the at least one position sensor includes an acoustic sensor configured to capture at least one acoustic image of the subject and relay the at least one acoustic image to the controller;
collecting one or more positional measurements includes capturing at least one acoustic image of the subject; and
correlating each of the one or more positional measurements with at least one reference location stored in memory includes,
correlating the at least one acoustic image with the at least one reference location stored in the memory; and
determining if the at least one electrode is within a distance stored in the memory as specified for effective use of the at least one electrode.

62. (canceled)

63. The method of claim 44, wherein the at least one position sensor includes a topography sensor configured to capture one or more sets of topographical data of the subject and relay the one or more sets of topographical data to the controller;
collecting one or more positional measurements includes capturing one or more sets of topographical data of the subject; and
correlating each of the one or more positional measurements with at least one reference location stored in memory includes,
correlating the one or more sets of topographical data of the subject with the at least one reference location stored in the memory; and
determining if the at least one electrode is within a distance stored in the memory as specified for effective use of the at least one electrode.

64. The method of claim 44, wherein the wherein the at least one position sensor includes at least one accelerometer configured to determine one or more changes in position of the at least one position sensor from a first time to a second time;
collecting one or more positional measurements includes capturing one or more changes in position of the at least one position sensor; and
correlating each of the one or more positional measurements with at least one reference location stored in memory includes,
correlating the one or more changes in position of the at least one position sensor with the at least one reference location stored in the memory; and
determining if the at least one electrode is within a distance stored in the memory as specified for effective use of the at least one electrode.

65. The method of claim 64, wherein correlating the one or more changes in position of the at least one position sensor with the at least one reference location stored in the memory includes determining the position of the at least one electrode based on a known start position and the one or more changes in position.

66. The method of claim 64, further including taking one or more electrical potential measurements with the at least one electrode.

67. The method of claim 66, further including tagging each of the one or more of the one or more electrical potential measurements with data regarding motion of the at least one electrode during the one or more electrical potential measurement.

68. The method of claim 66, further including rejecting at least one of the one or more electrical potential measurements if the one or more changes in position of the position sensor during the at least one of one or more electrical potential measurements exceeds a specified threshold.

69. The method of claim 44, further including,
collecting one or more electrical potential measurements with the at least one electrode; and
tagging each of the one or more of the one or more electrical potential measurements with data regarding the position of the at least one electrode during the one or more electrical potential measurements.

70. The method of claim 44, wherein providing an indication regarding a position of the at least one electrode relative to the at least one preferred measurement site includes indicating a desired motion of the at least one glove relative to the at least one preferred measurement site.

71. (canceled)

72. (canceled)

73. (canceled)
